(19) United States
(12) Patent Application Publication
(10) Pub. No.: US 2013/0310431 A1
(43) Pub. Date: Nov. 21, 2013

(54) USES FOR SUBSTITUTED 2-AMINO-THIAZOLONES IN TREATING ALZHEIMER'S DISEASE

(71) Applicant: Michele Wyszynski, Westlake Village, CA (US)

(72) Inventor: Michele Wyszynski, Westlake Village, CA (US)

(73) Assignee: Amgen Inc

(21) Appl. No.: 13/879,229

(22) PCT Filed: Oct. 11, 2011
(86) PCT No.: PCT/US11/55682

§ 371 Date: Aug. 2, 2013

Related U.S. Application Data
(60) Provisional application No. 61/393,445, filed on Oct. 15, 2010.

(51) Int. Cl. A61K 31/426

(52) U.S. Cl. CPC A61K 31/426
USPC 514/369; 548/184

(57) ABSTRACT

The invention provides 11-β-hydroxysteroid dehydrogenase type 1 enzyme (11βHSD1) inhibitors, such as a compound of the formula: (A), its hydroxy and keto metabolites, and pharmaceutically acceptable salts thereof, for treating cognitive disorders, including age-related cognitive disorders, such as Alzheimer's Disease. Also provided are methods and uses for inhibiting 11βHSD1 and for reducing the concentration or preventing the elevation of concentration of cortisol in the brain of a subject by administering the compound of the formula (A); its hydroxy and keto metabolites, and pharmaceutically acceptable salts thereof.

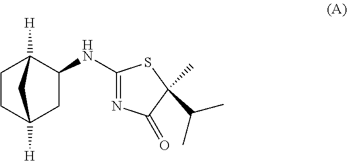

### BACKGROUND OF THE INVENTION

The present invention relates generally to substituted 2-amino-thiazolone inhibitors of 11-β-hydroxysteroid dehydrogenase type 1 enzyme (11βHSD1), as therapeutics for use in treating and preventing cognitive disorders, including depression, anxiety, schizophrenia, bipolar Disorder, post-traumatic stress disorder, ADHD, and Alzheimer's Disease.

Hydroxysteroid dehydrogenases (HSDs) regulate the occupancy and activation of steroid hormone receptors by converting steroid hormones into their inactive metabolites. For a review, see Nobel et al., Eur. J. Biochem. 268 (2001) 4113-4125.

There exist numerous classes of HSDs. The 11-beta-hydroxysteroid dehydrogenases (11β-HSDs) catalyze the interconversion of active glucocorticoids (such as cortisol and corticosterone), and their inert forms (such as cortisone and 11-dehydrocorticosterone). The isoform 11-beta-hydroxysteroid dehydrogenase type 1 (11β-HSD1) is expressed in liver, adipose tissue, brain, lung and other glucocorticoid tissue and is a potential target for therapy directed at numerous disorders that can be ameliorated by reduction of glucocorticoid action, such as age-related cognitive dysfunction. Seckl, et al., Endocrinology, 142 (2001) 1371-1376. See also Seckl et al., Trends Endocrinol. Metab. 15(9) (2004) 418-424.

In humans, the 11β-HSD catalyzes the conversion of cortisol to cortisone, and vice versa. The parallel function of 11β-HSD in rodents is the interconversion of corticosterone and 11-dehydrocorticosterone. See Frey, F. J., Escher, G., Frey, B. M. Pharmacology of 11 beta-hydroxysteroid dehydrogenase, Steroids 59(2) (1994) 74-9. Two isoenzymes of 11β-HSD, 11β-HSD1 and 11β-HSD2, have been characterized, and differ from each other in function and tissue distribution (Albiston, A. L., Obeyesekere, V. R., Smith, R. E., Krozowski, Z. S. Cloning and tissue distribution of the human 11 beta-hydroxysteroid dehydrogenase type 2 enzyme. Mol Cell Endocrinol 1994; 105(2):R11-7). Like glucocorticoid receptor (GR), 11β-HSD1 is expressed in numerous tissues like liver, adipose tissue, adrenal cortex, gonads, lung, pituitary, brain, eye etc (Monder C, White P C. 11 beta-hydroxysteroid dehydrogenase. Vitam Horm 1993; 47:187-271; Stewart, P. M., Krozowski, Z. S. 11 beta-Hydroxysteroid dehydrogenase. Vitam Horm 1999; 57:249-324; Stokes, J., Noble, J., Brett, L., Phillips, C., Seckl, J. R., O'Brien, C., et al. Distribution of glucocorticoid and mineralocorticoid receptors and 11beta-hydroxysteroid dehydrogenases in human and rat ocular tissues. Invest Ophthalmol Vis Sci 2000; 41(7):1629-38). The function of 11β-HSD1 is to fine-tune local glucocorticoid action.

In particular, evidence in the art implicates increasing levels of cortisone in cerebral spinal fluid (CSF), which is characteristic of Cushing's Disease patients, with decreasing cognitive function. The levels are least in subjects with subjective cognitive impairment, and increasingly greater in subjects with stable mild cognitive impairment, progressive mild cognitive impairment, and greatest in subjects with Alzheimer's Disease. See Gil-Bea et al., J. Alzheimer's Dis. 22(3) (2010). See also Hatzinger et al., Neurobiol. Aging 16(2) (1995) 205-209 and Elgh et al., Biol. Psychiatry 59(2) (2006) 155-161.

Further relevant to Alzheimer's Disease is the observation that hippocampal formation volume is inversely proportional to the concentration of urinary free cortisol, i.e., a decrease in cortisol reverses human hippocampal atrophy following treatment of Cushing's disease. See Starkman et al., Biol. Psychiatry 46(12) (1999) 1595-1602. It was also observed that cortisol normalization improves brain glucose homeostasis in patients with Cushing's Disease, suggesting that cerebral glucose metabolism can contribute to the cognitive and psychiatric abnormalities that are frequently observed in such patients. See Brunetti et al., J. Nucl. Med. 39(5) (1998) 786-790.

The enzyme 11β-HSD-1 is prominently expressed in the brain and catalyzes the formation of active 11-hydroxy steroids, such as cortisol, that are associated with cognitive disorders like Alzheimer's Disease. See Seckl (2001) and Seckl (2004).

Stress and glucocorticoids influence cognitive function (de Quervain, D. J.-F., B. Roozendaal, and J. L. McGaugh (1998) Nature 394: 787-790). The enzyme 11βHSD1 controls the level of glucocorticoid action in the brain and thus contributes to neurotoxicity (Rajan, V., C. R. W. Edwards, and J. R. Seckl, J. (1996) Neuroscience 16: 65-70; Seckl, J. R., Front. (2000) Neuroendocrinol. 18: 49-99). Based the above and on the known effects of glucocorticoids in the brain, inhibiting 11βHSD1 in the brain can result in reduced anxiety (Tronche, F. et al. (1999) Nature Genetics 23: 99-103). Thus, inhibition of 11βHSD1 in the human brain can prevent reactivation of cortisone into cortisol and protect against deleterious glucocorticoid-mediated effects on neuronal survival and other aspects of neuronal function, including cognitive impairment, depression, and increased appetite. Accordingly, inhibition of the enzyme constitutes an attractive therapeutic strategy for treating and preventing the disorders described herein.

### SUMMARY OF THE INVENTION

The invention provides in one embodiment a method for the treatment or prevention of an age-related cognitive disorder in a subject. The method prescribes administering to the subject a therapeutically effective amount of at least one compound selected from the group consisting of:

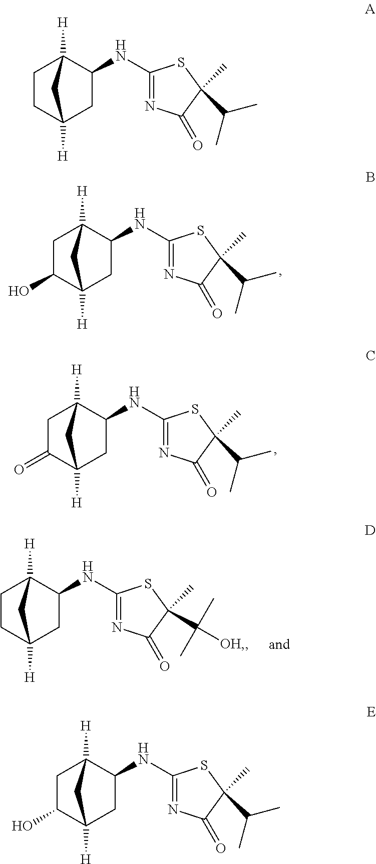

or pharmaceutically acceptable salts thereof.

The invention also provides a method for inhibiting 11β-hydroxysteroid dehydrogenase type 1 in the brain of a subject, comprising administering to the subject a therapeutically effective amount of at least one compound selected from the group consisting of A, B, C, D, and E,or a pharmaceutically acceptable salt thereof as described hereinabove.

Another embodiment of the invention is a method for reducing the concentration or preventing the elevation of concentration of cortisol in the brain of a subject. According to this embodiment, the method comprises administering to the subject a therapeutically effective amount of at least one compound selected from the group consisting of A, B, C, D, and E, or a or pharmaceutically acceptable salt as described hereinabove.

In addition, the invention provides a compound selected from the group consisting of A, B, C, D, and E, or a or pharmaceutically acceptable salt thereof as described hereinabove for the treatment or prevention of an age-related cognitive disorder in a subject, for inhibiting 11β-hydroxysteroid dehydrogenase type 1 in the brain of a subject, and/or for reducing the concentration or preventing the elevation of concentration of cortisol in the brain of a subject.

The invention provides in another embodiment a use of the compound selected from the group consisting of A, B, C, D, and E, or a or pharmaceutically acceptable salt thereof in the manufacture of a medicament for the treatment or prevention of an age-related cognitive disorder in a subject, for inhibiting 11β-hydroxysteroid dehydrogenase type 1 in the brain of a subject, and/or for reducing the concentration or preventing the elevation of concentration of cortisol in the brain of a subject.

In any of the embodiments described herein, the subject can be a human.

In another embodiment, optionally in combination with any other embodiment set forth herein, the subject suffers from a cognitive disorder, such as an age-related cognitive disorder. For instance, the cognitive disorder is selected from the group consisting of depression, anxiety, schizophrenia, bipolar disorder, post-traumatic stress disorder, attention deficit hyperactivity disorder (ADHD), and Alzheimer's Disease. An specific disorder is Alzheimer's Disease.

In combination with any other embodiment set forth herein, the compound can be:

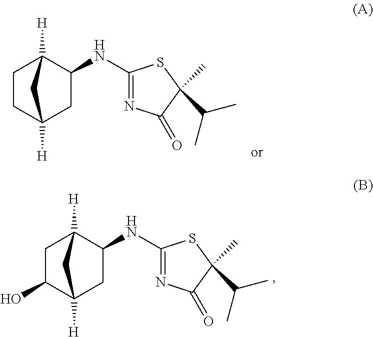

or a pharmaceutically acceptable salt thereof.

### BRIEF DESCRIPTION OF THE DRAWINGS

FIG. 1 shows the effects of a pharmaceutical vehicle (Veh), galantamine (Gal), and Compound (A) (AMG221)on the reference index of young and aged Wistar rats in novel object recognition study, showing that AMG221 reverses age-related behavioral deficits.

FIG. 2 presents pharmacokinetic data for Compound (A) (AMG221) as determined in cynomolgus monkey plasma, in brain regions, and in fat.

FIG. 3 presents pharmacokinetic data for Compound (B) as determined in cynomolgus monkey plasma, in brain regions, and in fat.

### DETAILED DESCRIPTION

The invention provides methods and uses for treating cognitive disorders, specifically age-related cognitive disorders, such as Alzheimer's Disease, by administration of substituted 2-amino-thiazolone inhibitors of 11β-HSD-1.

The methods and uses prescribe administration of at least one substituted 2-amino-thiazolone 11β-HSD-1 inhibitor to a subject, such as a human. In other embodiments, the subject is a mammal, such as a companion animal like a dog or cat. In other embodiments, the mammal is a horse.

In one embodiment, the inhibitor is a compound of the formula:

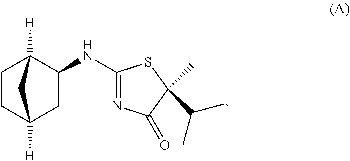

the synthesis of which is known in the art. See U.S. Pat. No. 7,253,196; U.S. Pat. No. 7,541,474; WO 2009/002445; and WO 2010/008729.

Alternatively, the inhibitor is a hydroxy or keto derivative of the compound above, selected from the group consisting of:

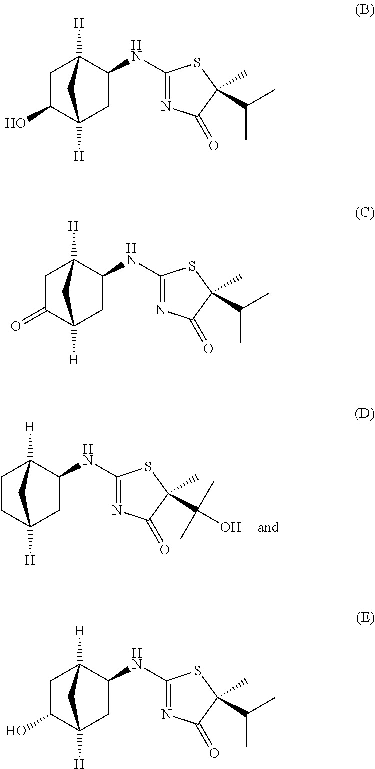

The hydroxy and keto derivatives, as well as their syntheses, are described in WO 2007/061661.

The substituted 2-amino-thiazolone 11β-HSD-1 inhibitors described herein are also suitable as their pharmaceutically acceptable salts for the inventive methods and uses. The expression “pharmaceutically acceptable” refers to the suitability of a compound or salt in preparation of a pharmaceutical composition that is generally safe, non-toxic and neither biologically nor otherwise undesirable, and it contemplates veterinary and human pharmaceutical use.

“Pharmaceutically acceptable salts” mean pharmaceutically acceptable substituted 2-amino-thiazolone 11β-HSD-1 salts, as defined above, which possess the desired pharmacological activity. Such salts include acid addition salts formed with organic and inorganic acids, such as hydrogen chloride, hydrogen bromide, hydrogen iodide, sulfuric acid, phosphoric acid, acetic acid, glycolic acid, maleic acid, malonic acid, oxalic acid, methanesulfonic acid, trifluoroacetic acid, fumaric acid, succinic acid, tartaric acid, citric acid, benzoic acid, ascorbic acid and the like. Base addition salts are formed with organic and inorganic bases, such as sodium, ammonia, potassium, calcium, ethanolamine, diethanolamine, N-methylglucamine, choline and the like. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. Furthermore, a pharmaceutically acceptable salt can have more than one charged atom in its structure. In this instance the pharmaceutically acceptable salt can have multiple counterions. Hence, a pharmaceutically acceptable salt can have one or more charged atoms and/or one or more counterions.

The substituted 2-amino-thiazolone 11β-HSD-1 inhibitors according to the present invention are also useful in the inventive methods and uses as pharmaceutical compositions. Pharmaceutical compositions according to the present invention contain a pharmaceutically acceptable carrier together with at least one of the substituted 2-amino-thiazolone 11β-HSD-1 inhibitors as described herein, and dissolved or dispersed therein as an active ingredient.

The preparation of a pharmaceutical composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The composition can also be emulsified.

The active ingredient can be mixed with excipients, which are pharmaceutically acceptable and compatible with the active ingredient and in amounts suitable for use in the therapeutic methods and uses described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. Adjuvants may also be present in the composition.

Pharmaceutically acceptable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. In addition, aqueous carriers can contain more than one buffer salt, as well as salts such as sodium and potassium chlorides, dextrose, propylene glycol, polyethylene glycol and other solutes.

Liquid compositions can also contain liquid phases in addition to, or to the exclusion of, water. Illustrative liquid phases are glycerine, vegetable oils such as cottonseed oil, organic esters such as ethyl oleate, and water-oil emulsions.

The substituted 2-amino-thiazolone compounds and their pharmaceutical compositions described herein can be administered orally, topically, intraperitoneally, intraarticularly, intracranially, intradermally, intramuscularly, intraocularly, intrathecally, intravenously, subcutaneously. Other routes are known to those of ordinary skill in the art.

Orally administrable compositions according to the present invention can be in the form of tablets, capsules, powders, granules, lozenges, liquid or gel preparations, such as oral, topical or sterile parenteral solutions or suspensions. Tablets and capsules for oral administration can be in unit dose presentation form and can contain conventional excipients such as binding agents, for example syrup, acacia, gelatin, sorbitol, traganath or polyvinyl-pyrrolidone; fillers e.g. lactose, sugar, maize-starch, calcium phosphate, calcium hydrogen phosphate, sodium starch glycolate, sorbitol or glycine; tabletting lubricant e.g. magnesium stearate, talc, polyethylene glycol or silicon dioxide (optionally colloidal); disintegrants e.g. potato starch, or acceptable wetting agents such as sodium lauryl sulfate. The tablets can be coated according to methods well known in normal pharmaceutical practice.

Oral liquid preparations can be in the form of aqueous or oily suspensions, solutions, emulsions, syrups or elixirs or as a dry product for reconstitution with water or other suitable vehicle before use. Such liquid preparations can contain conventional additives such as suspending agents, e.g. sorbitol, syrup, methyl cellulose (optionally microcrystalline), glucose syrup, gelatin hydrogenated edible fats; emulsifying agents like lecithin, sorbitan monooleate or acacia, non-aqueous vehicles (which may include edible oils), e.g. almond oil, fractionated coconut oil, oily esters such as glycerine, propylene glycol, or ethyl alcohol; preservatives e.g. methyl or propyl p-hydroxybenzoate or sorbic acid, and if desired conventional flavoring or coloring agents.

“An effective amount” refers to an amount of a substituted 2-amino-thiazolone compound which confers a therapeutic effect on the treated subject. The therapeutic effect may be objective (i.e., measurable by some test or marker) or subjective (i.e., subject gives an indication of or feels an effect). A pharmaceutical composition according to the present invention, may comprise typically an amount of at least 0.1 weight percent of compound comprising the formula (I) per weight of total therapeutic composition. A weight percent is a ratio by weight of total composition. Thus, for example, 0.1 weight percent is 0.1 grams of compound comprising the formula (I) per 100 grams of total composition. A suitable daily oral dose for a mammal, preferably a human being, may vary widely depending on the condition of the patient. However a dose of compound comprising the substituted 2-amino-thiazolone of about 0.1 to 300 mg/kg body weight may be appropriate.

The compositions according to the present invention can also be used veterinarily and thus they may comprise a veterinarily acceptable excipient or carrier. The substituted 2-amino-thiazolone compounds and compositions may be thus administered to animals, e.g., cats, dogs, or horses, in treatment methods.

The substituted 2-amino-thiazolone compounds described herein can be administered in the form of suppositories for rectal administration. These suppositories can be prepared by mixing the drug with a suitable non-irritating excipient which is solid at ordinary temperatures but liquid at the rectal temperature and will therefore melt in the rectum to release the drug. Such excipients include cocoa butter and polyethylene glycols.

Pharmaceutical compositions for parenteral administrations are administered in a sterile medium. Depending on the vehicle used and the concentration of the substituted 2-amino-thiazolone compound in the formulation, the parenteral formulation can either be a suspension or a solution containing the dissolved compound. Adjuvants such as local anesthetics, preservatives and buffering agents can also be added to parenteral compositions.

The substituted 2-amino-thiazolone compounds and pharmaceutical compositions described herein are useful for administration to a subject as prescribed by the inventive methods and uses. Typical dosage levels generally range from about 0.001 to about 100 mg per kg patient body weight per day which can be administered in single or multiple doses. An exemplary dosage is about 0.01 to about 25 mg/kg per day or about 0.05 to about 10 mg/kg per day. In other embodiments, the dosage level is from about 0.01 to about 25 mg/kg per day, about 0.05 to about 10 mg/kg per day, or about 0.1 to about 5 mg/kg per day.

A dose typically ranges from about 0.1 mg to about 2000 mg per day, given as a single once-a-day dose or, alternatively, as divided doses throughout the day, optionally taken with food. In one embodiment, the daily dose is administered twice daily in equally divided doses. A daily dose range can be from about 5 mg to about 500 mg per day, such as, for example, between about 10 mg and about 300 mg per day. In managing the patient, the therapy can be initiated at a lower dose, perhaps from about 1 mg to about 25 mg, and increased if necessary from about 200 mg per day to about 2000 mg per day, administered as either a single dose or multiple doses, depending on the patient's global response.

All publications cited herein are hereby incorporated by reference as if fully set forth in their entireties.

The invention will now be further described in reference to the following Examples. These Examples are not to be regarded as limiting the scope of the present invention, but shall only serve in an illustrative manner.

### EXAMPLES

### Example 1

### Enzyme Assays

A. Scintillation Proximity Assay (SPA)

[1, 2(n)-³H]-cortisone was purchased from Amersham Pharmacia Biotech. Anti-cortisol monoclonal mouse antibody, clone 6D6.7 was obtained from Immunotech and Scintillation proximity assay (SPA) beads coated with monoclonal antimouse antibodies were from Amersham Pharmacia Biotech. NADPH, tetrasodium salt was from Calbiochem and glucose-6-phosphate (G-6-P) was supplied by Sigma. The human 11-β-hydroxysteroid dehydrogenase type-1 enzyme (11-β-HSD₁) was expressed in Pichia pastoris. 18-β-glycyrrhetinic acid (GA) was obtained from Sigma. The serial dilutions of the compounds were performed on a Tecan Genesis RSP 150. Compounds to be tested were dissolved in DMSO (1 mM) and diluted in 50 mM Tris-HCl, pH 7.2 containing 1 mM EDTA.

The multiplication of plates was done on a WallacQuadra. The amount of the product [³H]-cortisol, bound to the beads was determined in a Packard, Top Count microplate liquid scintillation counter.

The 11-β-HSD₁ enzyme assay was carried out in 96 well microtiter plates (Packard, Optiplate) in a total well volume of 220 μL and contained 30 mM Tris-HCl, pH 7.2 with 1 mM EDTA, a substrate mixture tritiated Cortisone/NADPH (175 nM/181 μM), G-6-P (1 mM) and inhibitors in serial dilutions (9 to 0.15 μM). Reactions were initiated by the addition of human 11-β-HSD₁, either as Pichia pastoris cell homogenate or microsomes prepared from Pichia pastoris (the final amount of enzyme used was varied between 0.057 to 0.11 mg/mL). Following mixing, the plates were shaken for 30 to 45 minutes at room temperature. The reactions were terminated with 10 μL 1 mM GA stop solution. Monoclonal mouse antibody was then added (10 μL of 4 μM) followed by 100 μL of SPA beads (suspended according to the manufacturers instructions). Appropriate controls were set up by omitting the 11-β-HSD₁ to obtain the non-specific binding (NSB) value.

The plates were covered with plastic film and incubated on a shaker for 30 minutes, at room temperature, before counting. The amount of [³H]-cortisol, bound to the beads was determined in a microplate liquid scintillation counter. The calculation of the Kᵢ values for the inhibitors was performed by use of Activity Base. The Kᵢ value is calculated from IC₅₀ and the Kₘ value is calculated using the Cheng Prushoff equation (with reversible inhibition that follows the Michaelis-Menten equation): Kᵢ=IC₅₀(1+[S]/Kₘ) [Cheng, Y. C.; Prushoff, W. H. Biochem. Pharmacol. 1973, 22, 3099-3108]. The IC₅₀ is measured experimentally in an assay wherein the decrease of the turnover of cortisone to cortisol is dependent on the inhibition potential of an inhibitor.

The expression and purification of the murine enzyme is described by J. Zhang, et al. Biochemistry 44 (2005) 6948-57. The expression and purification of the human enzyme is similar to that of the murine sequence.

The procedures described above yielded the following results for the inhibitors described herein:

| | | SPA Kᵢ | Whole | |
|---|---|---|---|---|
| | | avg. | Cell IC₅₀ | Relative |
| Compound | | (μM) | (μM) | Potency |
| 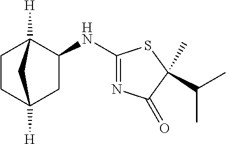 | (A) | 0.009 | 0.009 | 1 |
| 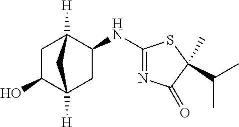 | (B) | 0.018 | 0.052 | 0.2 |
| 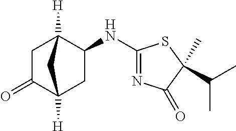 | (C) | 0.015 | 0.015 | 0.6 |
| 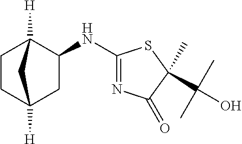 | (D) | 0.015 | 0.007 | 1.3 |
| 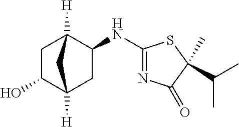 | (E) | 0.019 | 0.010 | 1.0 |

### Example 2

### Reversal of Age-Related Behavioral Deficits

This example demonstrates the superior efficacy of compound AMG221 in treating behavioral deficits in aged rats, as determined by a novel object recognition study. Thus, over a 14 day treatment period, young and aged (20 months) Wistar rats were administered formulations as follows:

1. Young: pharmaceutical vehicle (no active agent);

2. Young: galantamine, an acetylcholinesterase inhibitor as a positive control;

3. Aged: vehicle;

4. Aged: galantamine;

5. Aged: compound AMG221 (3 mg/kg);

6. Aged: compound AMG221 (10 mg/kg); and

7. Aged: compound AMG221 (30 mg/kg).

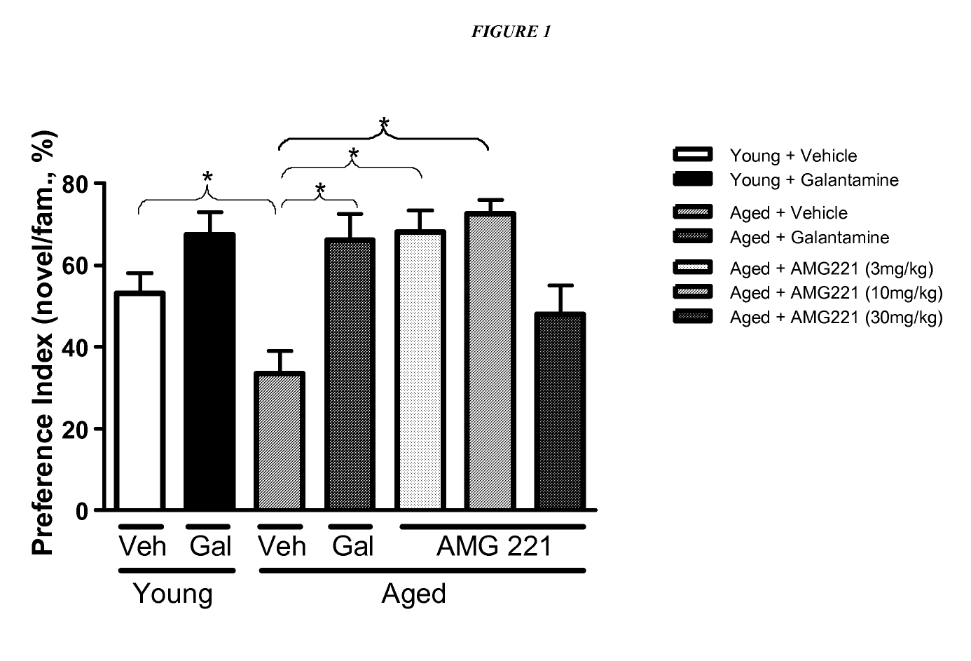

Aged rats that were administered compound AMG221 exhibited superior improvement in novel object recognition, when compared to rats that had been administered a commercialized Alzheimer's Disease drug, galantamine (FIG. 1).

### Example 2

### Inhibition of 11β-HSD1 in Non-Human Primate Brain

The purpose of this example is to demonstrate that compound AMG221 and its hydroxy metabolite, (B),

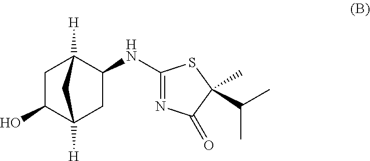

permeate highly into brain tissue and inhibit 11β-HSD1 in the brain.

In general, cynomolgus monkeys were separately administered AMG221 (A) and (B) by intravenous infusion for 1 hour at concentrations to deliver multiples of Kᵢ for 11β-HSD1. Samples of brain tissue from regions with high and low 11β-HSD1 expression were collected, along with samples of cerebrospinal fluid (CSF), plasma, and fat. Concentrations of AMG221 and (B), and their inhibition of 11β-HSD1, were measured for each sample.

A. AMG221 Concentration vs. Time Profiles

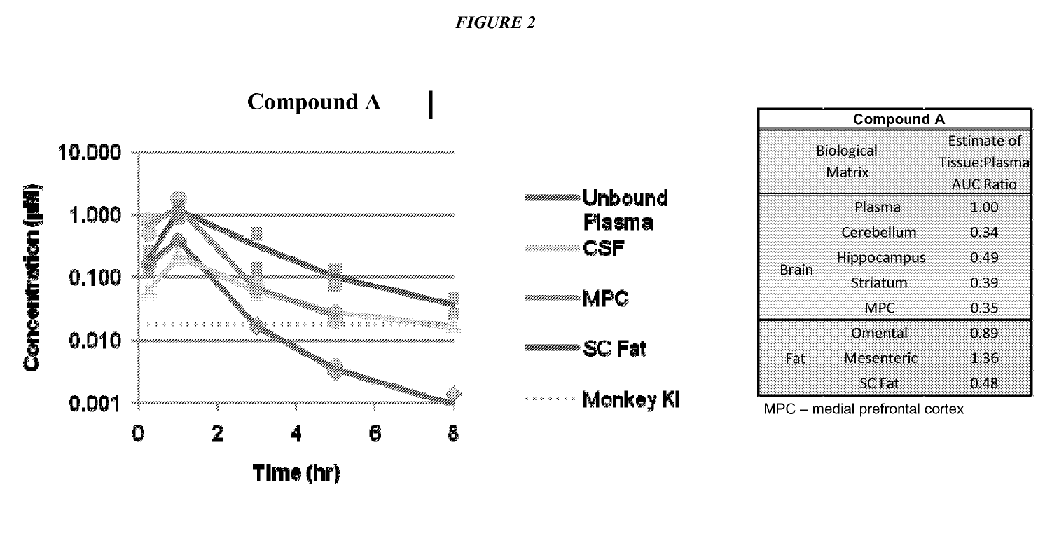

The concentration of AMG221 in the biological samples described above was measured as a function of time. As shown in FIG. 2, concentrations of AMG221 exceeded the cynomolgus Kᵢ for 11β-HSD1 in unbound plasma for three hours and in CSF for eight hours. The results indicate that AMG221 distributed uniformly and more rapidly within the central nervous system than in fat.

B. Compound(B) Concentration vs. Time Profiles

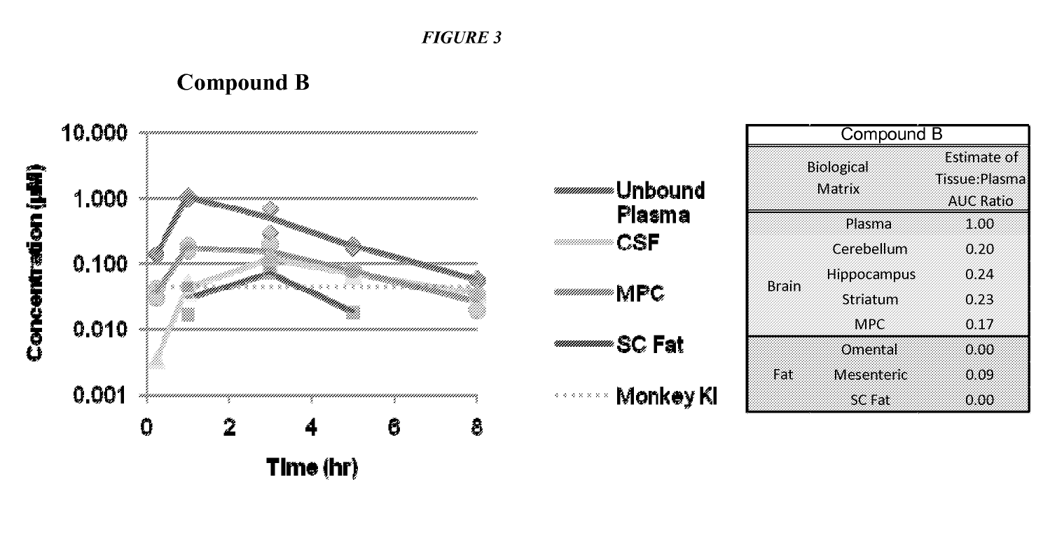

The concentration of (B) in the biological samples described above was measured as a function of time. As shown in FIG. 3, concentrations of compound (B) exceeded the cynomolgus Kᵢ for 11β-HSD1 in unbound plasma for eight hours and in CSF for five hours. The results indicate that compound (B) distributed uniformly and more rapidly within the central nervous system than in fat. In addition, the results show that compound B distributed poorly into fat, relative to AMG221.

These data suggest that inhibition of 11β-HSD1 in the brain can be predicted based upon the pharmacokinetic (PK) and pharmacodynamic (PD) parameters in fat.

C. Inhibition of 11β-HSD1 in Non-Human Primate Brain

An ex vivo assay on cynomolgus monkey brains assessed the extent of 11β-HSD1 inhibition by AMG221 by measuring the extent to which administered levels of the synthetic corticosteroid prednisone were converted to prednisolone Inhibition data for the medial prefrontal cortex demonstrated that AMG221 effected greater than 90% inhibition of 11β-HSD1 activity from 0.25 through 5 h after treatment with AMG221. In addition, cynomolgus cerebellum data demonstrated that greater than 90% inhibition also occurred from 0.25 through 8 h. Both sets of data were based upon a prednisolone lower limit of quantification (LLOQ) of 0.25 ng/mL, which was equivalent to the rate of 1.9 pmoles/g/hr.

An analogous assay in cynomolgus fat demonstrated greater than 80% inhibition of 11β-HSD1 activity from 0.25 through 5 h, based upon a prednisolone LLOQ of 0.25 ng/mL (3.7 pmoles/g/hr). Accordingly, 11β-HSD1 inhibition in fat exhibited a similar pattern to that of the MPC and cerebellum data above.

These data demonstrate that AMG221 inhibits 11β-HSD1 in the brain.

We claim:

1. A method for the treatment or prevention of a cognitive disorder in a subject, comprising administering to the subject a therapeutically effective amount of at least one compound selected from the group consisting of: 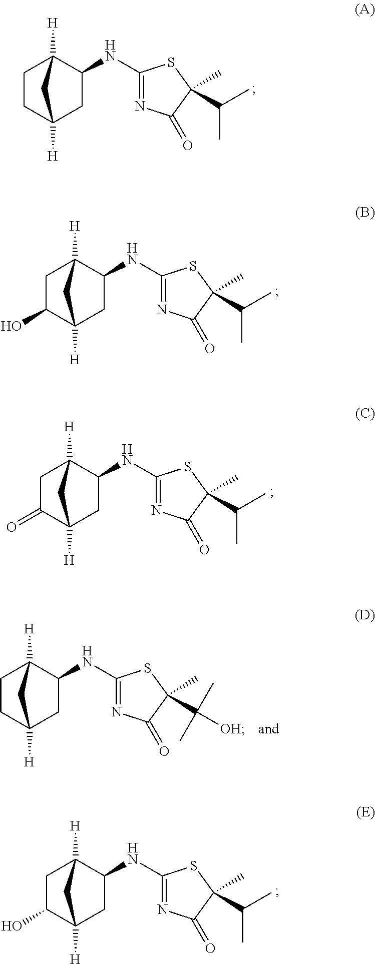
or pharmaceutically acceptable salts thereof.

2. The method according to claim 1, wherein the subject is a human.

3. The method according to claim 1, wherein the cognitive disorder is selected from the group consisting of depression, anxiety, schizophrenia, bipolar disorder, post-traumatic stress disorder, attention deficit hyperactivity disorder (ADHD), and Alzheimer's Disease.

4. The method according to claim 3, wherein the cognitive disorder is Alzheimer's Disease.

5. The method according to claim 1, wherein the compound is:

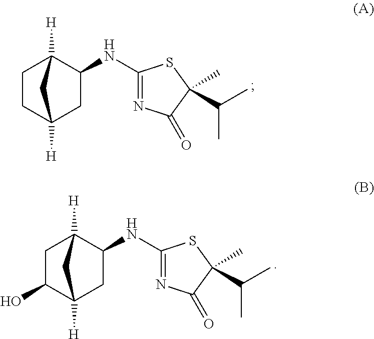

6. The method according to claim 1, wherein the cognitive disorder is Alzheimer's Disease and the compound is:

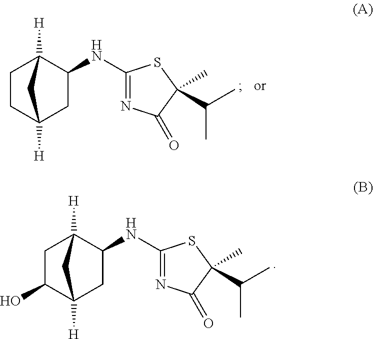

7. A method for inhibiting 11β-hydroxysteroid dehydrogenase type 1 in the brain of a subject, comprising administering to the subject a therapeutically effective amount of at least one compound selected from the group consisting of: 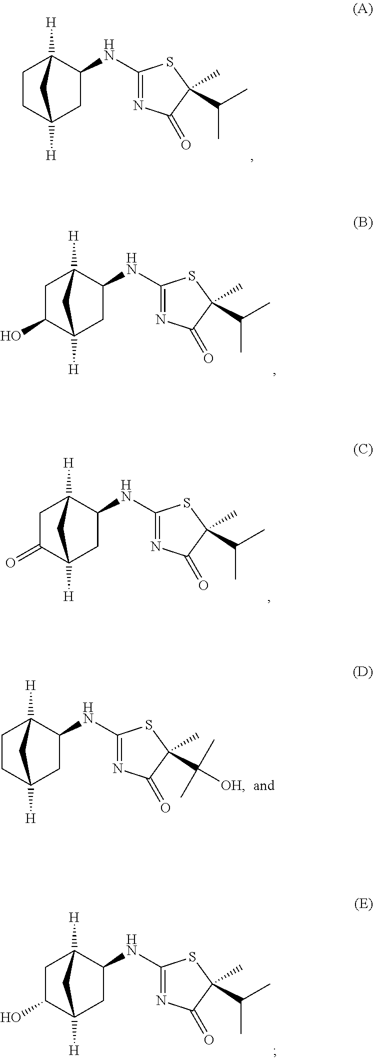
or pharmaceutically acceptable salts thereof.

8. The method according to claim 7, wherein the subject is human.

9. The method according to claim 7, wherein the subject suffers from a cognitive disorder selected from the group consisting of depression, anxiety, schizophrenia, bipolar disorder, post-traumatic stress disorder, attention deficit hyperactivity disorder (ADHD), and Alzheimer's Disease.

10. The method according to claim 9, wherein the cognitive disorder is Alzheimer's Disease.

11. The method according to claim 10, wherein the compound is:

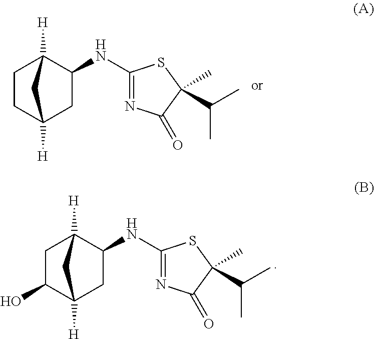

12. The method according to claim 7, wherein the cognitive disorder is Alzheimer's Disease and the compound is:

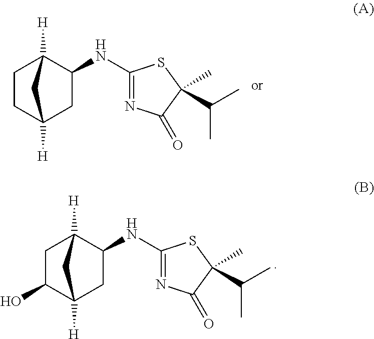

13. A method for reducing the concentration or preventing the elevation of concentration of cortisol in the brain of a subject, comprising administering to the subject a therapeutically effective amount of at least one compound selected from the group consisting of: 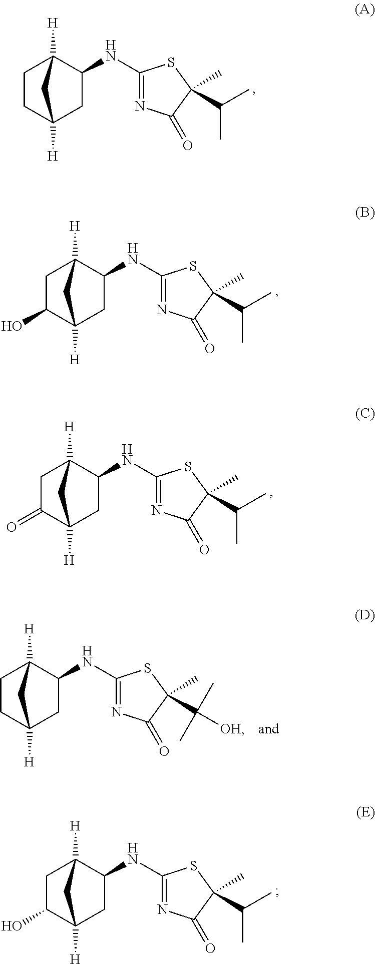
and pharmaceutically acceptable salts thereof.

14. The method according to claim 13, wherein the subject is a human.

15. The method according to claim 13, wherein the cognitive disorder is selected from the group consisting of depression, anxiety, schizophrenia, bipolar disorder, post-traumatic stress disorder, attention deficit hyperactivity disorder (ADHD), and Alzheimer's Disease.

16. The method according to claim 15, wherein the cognitive disorder is Alzheimer's Disease.

17. The method according to claim 13, wherein the compound is:

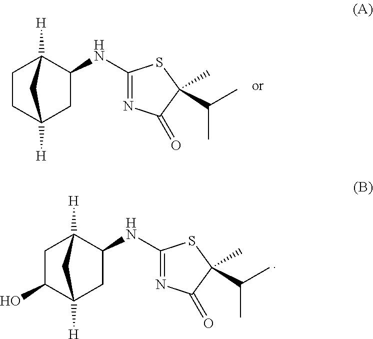

18. The method according to claim 13, wherein the cognitive disorder is Alzheimer's Disease and the compound is:

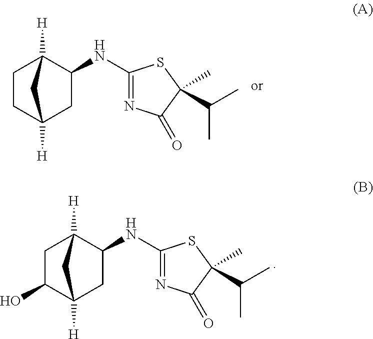

19. A compound selected from the group consisting of 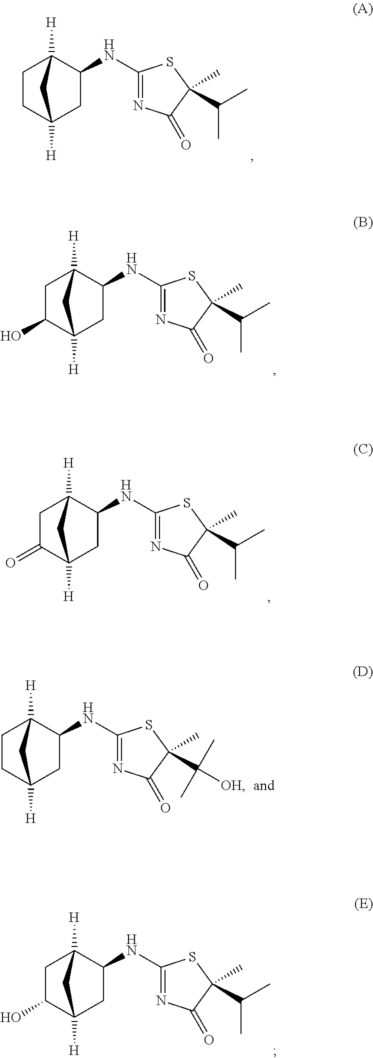
or pharmaceutically acceptable salts thereof, for the treatment or prevention of cognitive disorder in a subject, for inhibiting 11β-hydroxysteroid dehydrogenase type 1 in the brain of a subject, and/or for reducing the concentration or preventing the elevation of concentration of cortisol in the brain of a subject.

20. The compound according to claim 19, wherein the subject is human.

21. The compound according to claim 19, wherein the cognitive disorder is selected from the group consisting of depression, anxiety, schizophrenia, bipolar disorder, post-traumatic stress disorder, attention deficit hyperactivity disorder (ADHD), and Alzheimer's Disease.

22. The compound according to claim 21, wherein the cognitive disorder is Alzheimer's Disease.

23. The compound according to claim 21, wherein the compound is:

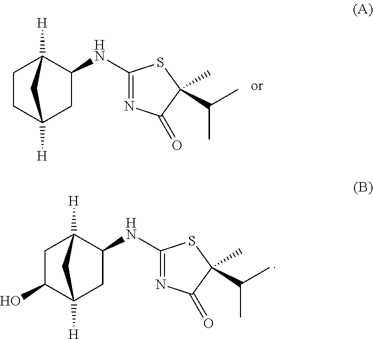

24. The compound according to claim 21, wherein the cognitive disorder is Alzheimer's Disease and the compound is:

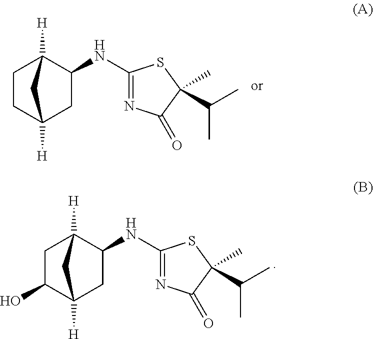

25. Use of a compound selected from the group consisting of 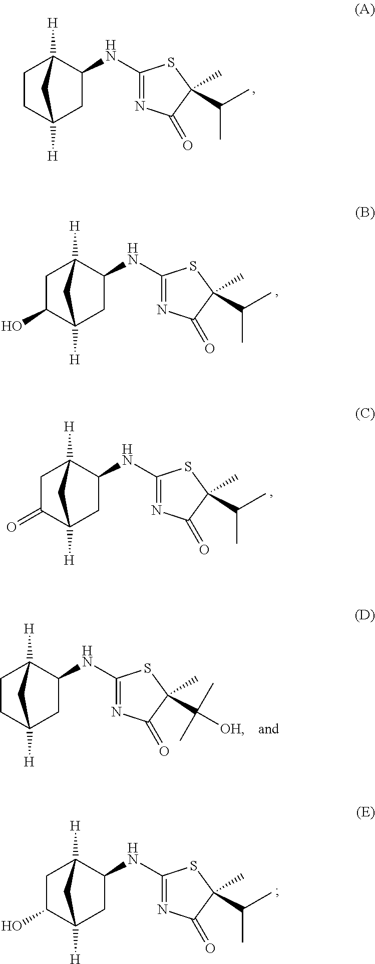
or pharmaceutically acceptable salts thereof, in the manufacture of a medicament for the treatment or prevention of cognitive disorder in a subject, for inhibiting 11β-hydroxysteroid dehydrogenase type 1 in the brain of a subject, and/or for reducing the concentration or preventing the elevation of concentration of cortisol in the brain of a subject.

26. The use according to claim 25, wherein the subject is human.

27. The use according to claim 25, wherein the cognitive disorder is selected from the group consisting of depression, anxiety, schizophrenia, bipolar disorder, post-traumatic stress disorder, attention deficit hyperactivity disorder (ADHD), and Alzheimer's Disease.

28. The use according to claim 27, wherein the cognitive disorder is Alzheimer's Disease.

29. The use according to claim 25, wherein the compound is:

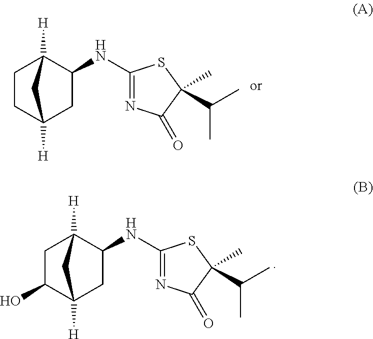

30. The use according to claim 25, wherein the cognitive disorder is Alzheimer's Disease and the compound is: [chemical structure]
